(19) United States
(12) Patent Application Publication
(10) Pub. No.: US 2019/0354990 A1
(43) Pub. Date: Nov. 21, 2019

(54) ADAPTING A VIRTUAL AGENT SUPPORT SYSTEM BASED ON EMOTIONAL STATE AND DEMOGRAPHICS INFORMATION

(71) Applicant: INTERNATIONAL BUSINESS MACHINES CORPORATION, Armonk, NY (US)

(72) Inventors: Craig M. Trim, Ventura, CA (US); John M. Ganci, JR., Cary, NC (US); Martin G. Keen, Cary, NC (US); James E. Bostick, Cedar Park, TX (US); Diman Ghazi, Vancouver (CA)

(21) Appl. No.: 15/981,281

(22) Filed: May 16, 2018

(51) Int. Cl. G06Q 30/00; A61B 5/16

(52) U.S. Cl. CPC G06Q 30/016; A61B 5/024; A61B 5/163; A61B 5/165

(57) ABSTRACT

A computer-implemented method includes: monitoring, by a computing device, a support dialogue between a user device and a live agent system associated with the computing device; determining, by the computing device, emotional state information associated with the support dialogue; generating, by the computing device, emotional state-driven mapping information by mapping a particular path, of a plurality of possible paths, in a business process followed by the live agent system during a start of the support dialogue to a conclusion of the support dialogue and linking the particular path to the emotional state information; storing, by the computing device, the emotional state-driven mapping information; accessing, by the computing device, the emotional state-driven mapping information by a virtual agent system associated with the computing device; providing, by the computing device, automated support by the virtual agent system for a future support dialogue using the emotional state-driven mapping information.

### BACKGROUND

The present invention generally relates to adapting a virtual support agent system and, more particularly, to adapting a virtual support agent system based on emotional state demographics information.

A user may use a user computer device to interact with a virtual support agent (e.g., a chat bot, automated support agent, or the like) to obtain information or assistance for a technical issue (e.g., to reset a password, make changes to a service account, etc.) or other type of support issue. A virtual support agent system (referred to as an “agent” or “agent system”) and a user may engage in a dialogue in which the agent asks questions to determine the user's issues and provide the user with a solution. Virtual support agents may be designed to follow a path in a business process for providing support based on queries received from the user, and provide the user with appropriate responses to those queries.

### SUMMARY

In an aspect of the invention, a computer-implemented method includes: monitoring, by a computing device, a support dialogue between a user device and a live agent system associated with the computing device; determining, by the computing device, emotional state information associated with the support dialogue; generating, by the computing device, emotional state-driven mapping information by mapping a particular path, of a plurality of possible paths, in a business process followed by the live agent system during a start of the support dialogue to a conclusion of the support dialogue and linking the particular path to the emotional state information; storing, by the computing device, the emotional state-driven mapping information; accessing, by the computing device, the emotional state-driven mapping information by a virtual agent system associated with the computing device; providing, by the computing device, automated support by the virtual agent system for a future support dialogue using the emotional state-driven mapping information.

In an aspect of the invention, there is a computer program product for adapting a virtual agent system to provide automated support based on emotional stated-driven information. The computer program product comprising a computer readable storage medium having program instructions embodied therewith, the program instructions executable by a computing device to cause the computing device to: monitor a support dialogue between a user device and a live agent system associated with the computing device; determine emotional state information associated with the support dialogue; generate emotional state-driven mapping information by mapping a particular path, of a plurality of possible paths, in a business process followed by the live agent system during a start of the support dialogue to a conclusion of the support dialogue and linking the particular path to the emotional state information; store the emotional state-driven mapping information; transfer the support dialogue to a virtual agent system associated with the computing device; access the emotional state-driven mapping information by a virtual agent system; and provide automated support by the virtual agent system for the transferred support dialogue using the emotional state-driven mapping information.

In an aspect of the invention, a system includes: a CPU, a computer readable memory and a computer readable storage medium associated with a computing device; program instructions to receive a support request from a user device, wherein the support request identifies a support topic; program instructions to determine whether emotional state-driven mapping information for a business process is available for the support topic; program instructions to generate the emotional state-driven mapping information when the emotional state-driven mapping information is not available, wherein the generating comprises: program instructions to route the support request to a live agent system, program instructions to determine emotional state information associated with the support dialogue, and program instructions to map a particular path, of a plurality of possible paths, in the business process followed by the live agent system during a start of the support dialogue to a conclusion of the support dialogue and linking the particular path to the emotional state information. The system further includes program instructions to provide, when the emotional state-driven mapping information is available, automated support using the emotional state-driven mapping information and based on emotional state data received from the user device. The program instructions are stored on the computer readable storage medium for execution by the CPU via the computer readable memory.

### BRIEF DESCRIPTION OF THE DRAWINGS

The present invention is described in the detailed description which follows, in reference to the noted plurality of drawings by way of non-limiting examples of exemplary embodiments of the present invention.

FIG. 1 depicts a cloud computing node according to an embodiment of the present invention.

FIG. 2 depicts a cloud computing environment according to an embodiment of the present invention.

FIG. 3 depicts abstraction model layers according to an embodiment of the present invention.

FIGS. 4A and 4B show an overview of an example implementation in accordance with aspects of the present invention.

FIG. 5 shows an example environment in accordance with aspects of the present invention.

FIG. 6 shows a block diagram of example components of a user assistance system in accordance with aspects of the present invention.

FIGS. 7A and 7B show example diagrams of a process for generating emotional state-based mapping information and adapting a virtual agent system using the emotional state-based mapping information in accordance with aspects of the present invention.

FIG. 8 shows an example flowchart of a process for generating emotional state-based mapping information in accordance with aspects of the present invention.

FIG. 9 shows an example flowchart of a process for adapting a virtual agent to provide support to a user based on the user's demographics and/or emotional state in accordance with aspects of the present invention.

### DETAILED DESCRIPTION

The present invention generally relates to adapting a virtual support agent system and, more particularly, to adapting a virtual support agent system based on emotional state demographics information. In many expert support systems, a common methodology or business process is followed for providing a user with support for a topic (e.g., information about a topic, answers to questions about the topic, instructions for completing a task, etc.). This methodology generally presupposes that the business process used by a subject matter expert (SME) during the course of business has a one to one transcription with the expert system. For example, a call center representative who deals with reset password issues may follow a relatively straight-forward business process that can be easily adopted as-is by a virtual agent. That is, a reset password process may not vary greatly from user to user. Regardless of whether the user is male or female, young or old, or angry or depressed or happy, the business process for assisting a user with a relatively simple issue (e.g., a password reset) may not vary greatly, and thus, can be easily adopted by a virtual agent.

There are, however, circumstances where the path taken in a business process may vary when an SME has a wide leeway in application of the business process to an end user. For example, in the matter of counselling an individual, there is not a one-size-fits-all approach in which the same path within a business process is followed. Counselling, or other complex issues, is not simply a matter of sticking to a linear script without regard to the demographical attributes and/or emotional state of the person who is on the receiving end of the counselling. In these cases, the path within a business process that the SME follows for providing support to the individual may vary widely, and the variation may be based on the demographic attributes and emotional state of the user. Accordingly, aspects of the present invention provide a system to adapt a virtual agent to navigate a business process in order to provide support with consideration to data, such as demographics data and/or emotional state data. In this way, a virtual agent may be utilized to provide a user with support in a wide variety of topics, thus reducing the amount of manual labor and the required by SMEs to provide the support to users. Further, a greater number of users can be served by the use of virtual agents rather than by live agents (e.g., live SMEs).

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

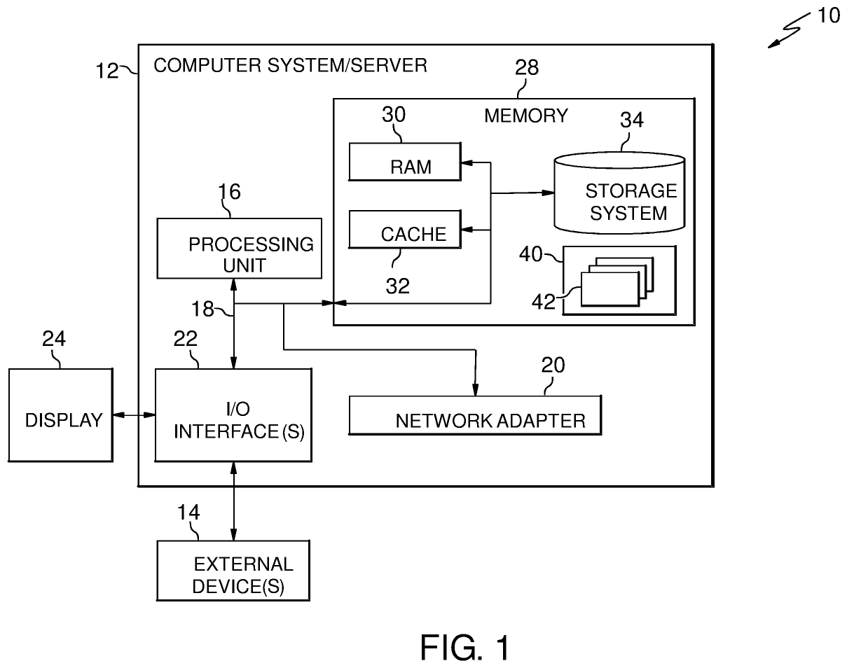

Referring now to FIG. 1, a schematic of an example of a cloud computing node is shown. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 1, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a nonremovable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

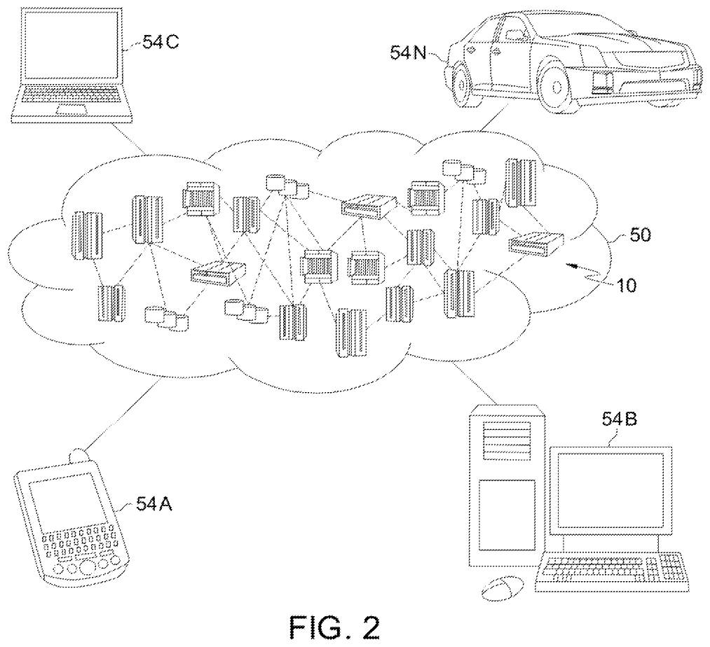

Referring now to FIG. 2, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 2 are intended to be illustrative only and that computing nodes 10 and cloud computing environment 50 can communicate with any type of computerized device over any type of network and/or network addressable connection (e.g., using a web browser).

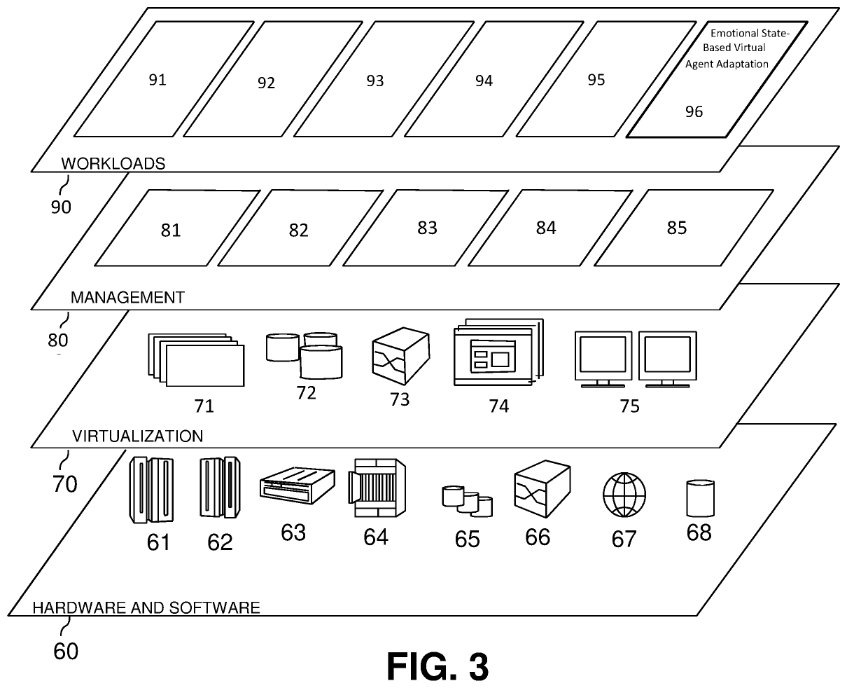

Referring now to FIG. 3, a set of functional abstraction layers provided by cloud computing environment 50 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below.

Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94;

transaction processing 95; and emotional state-based virtual agent adaptation 96.

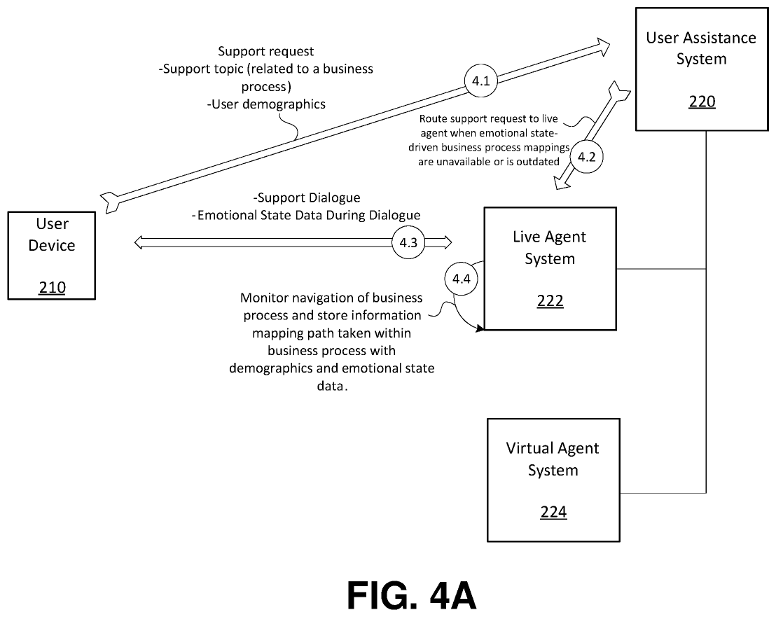

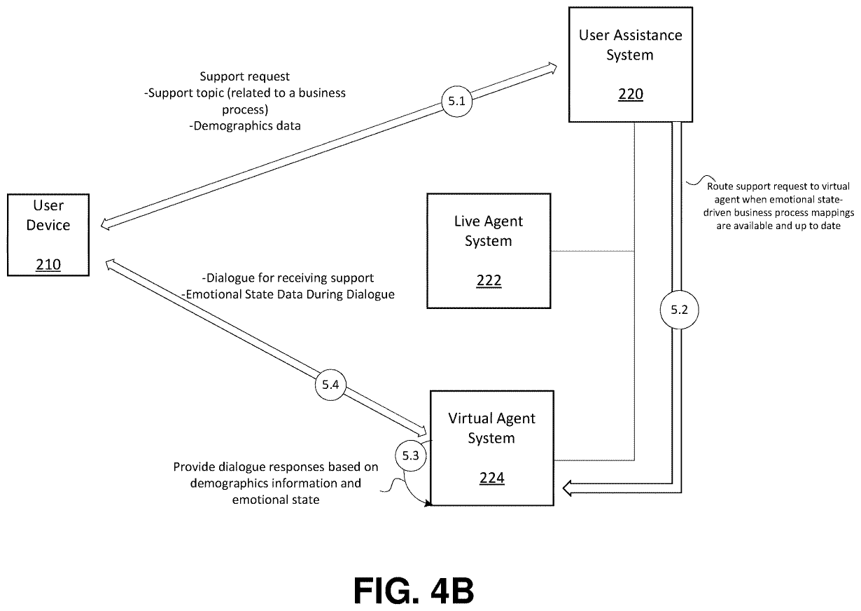

Referring back to FIG. 1, the program/utility 40 may include one or more program modules 42 that generally carry out the functions and/or methodologies of embodiments of the invention as described herein (e.g., such as the functionality provided by emotional state-based virtual agent adaptation 96). Specifically, the program modules 42 may monitor dialogue between a user and a live agent, determine the emotional state of the user during the dialogue, map a business process path from the beginning of the dialogue to the conclusion of the dialogue between the user and the live agent, store or update information mapping the emotional state at each business process step in the path, and adapt a virtual agent to follow the business process path associated with the user's emotional state for future dialogues between users in the emotional state. Other functionalities of the program modules 42 are described further herein such that the program modules 42 are not limited to the functions described above. Moreover, it is noted that some of the modules 42 can be implemented within the infrastructure shown in FIGS. 1-3. For example, the modules 42 may be representative of a user assistance system as shown in FIGS. 4A and 4B.

FIGS. 4A and 4B show an overview of an example implementation in accordance with aspects of the present invention. In FIGS. 4A and 4B, a user may use an application running on a user device 210 to receive support about a topic from a user assistance system 220. For example, the user may use the application to receive support (e.g., information about a topic, answers to questions about the topic, instructions for completing a task, etc.) via the user assistance system 220. The user assistance system 220 may include a support platform via which a live agent and/or virtual agent may provide support to the user (e.g., via a chat/instant messaging system, audio/video teleconference, etc.). As described herein, a support topic is related to a business process (e.g., a process for providing support to the user). In accordance with aspects of the present invention, emotional state-driven business process mappings may be determined and stored. The emotional state-driven business process mappings may include mapping information that maps the path taken (e.g., by a live agent) within a business process for providing the user with support based on the user's demographics and/or emotional state.

As shown in FIG. 4A, the user device 210 may provide a support request (at step 4.1) which may include user input identifying the support topic as well as the user's demographics information. At step 4.2, the user assistance system 220 may receive the request, and the user assistance system 220 may route the support request to a live agent when emotional state-driven business process mappings for the support topic are unavailable (e.g., not yet created) or are outdated (e.g., with respect to a configurable expiration time period). At step 4.3, a live agent, using a live agent system 222, may engage in a support dialogue in which the live agent provides support to the user in accordance with a business process associated with the support topic. During the dialogue, the live agent system 222 may receive emotional state data from the user device 210. For example, the user device 210 may provide biometrics data that is gathered by the user device 210 itself and/or via accessory devices connected to the user device 210 (e.g., smart watches, smart fitness bands, smart eye contacts, smart glasses, smart wearable computing devices, etc.). Additionally, or alternatively, the emotional state data may include audio/video data from the dialogue between the user and the live agent.

At step 4.4, the live agent system 222 may monitor the navigation of the business process and store information mapping a particular path, of multiple possible paths, taken within a business process with the demographics data and the emotional state data at each step in the business process. As described herein, the live agent system 222 may derive the emotional state of the user using the emotional state data and one or more suitable cognitive emotional state determination techniques. The live agent system 222 may monitor how a live agent navigated the business process from start to conclusion (e.g., the path taken in the business process). Further, the live agent system 222 may determine the emotional state of the user (e.g., at each step of the business process, and/or an aggregate emotional state of the user during the entire dialogue).

As described herein, the user assistance system 220 may store the emotional state-driven business process mappings (e.g., generated at step 4.4), and use the emotional state-driven business process mappings to adapt a virtual agent system 224 to provide a similar support experience (e.g., as did the live agent) to users who seek support in the future for the support topic (or even potentially during a support dialogue with a live agent in which the virtual agent takes over for the live agent). In this way, the virtual agent system 224 may provide users with support in a similar manner as did a live agent, factoring in emotional state data and/or demographics data. As such, live agent labor is saved, allowing a support provider to serve a larger number of users in a shorter amount of time. Further, aspects of the present invention adapt a virtual agent system 224 to provide a more seamless support experience that closely simulates the support experience provided by a live agent, thus improving user experience and satisfaction.

Referring to FIG. 4B, another user using a user device 210 may communicate with the user assistance system 220 to obtain support by providing a support request (e.g., at step 5.1). At step 5.2, the user assistance system 220 may determine that emotional state-driven business process mappings are available and up to date, and may route the support request to the virtual agent system 224. At steps 5.3 and 5.4, the virtual agent system 224 may engage in a dialogue with the user via the user device 210, determine the user's emotional state, and provide support to the user based on the emotional state-driven business process mappings stored by the user assistance system 220 (e.g., created at step 4.4 in FIG. 4A).

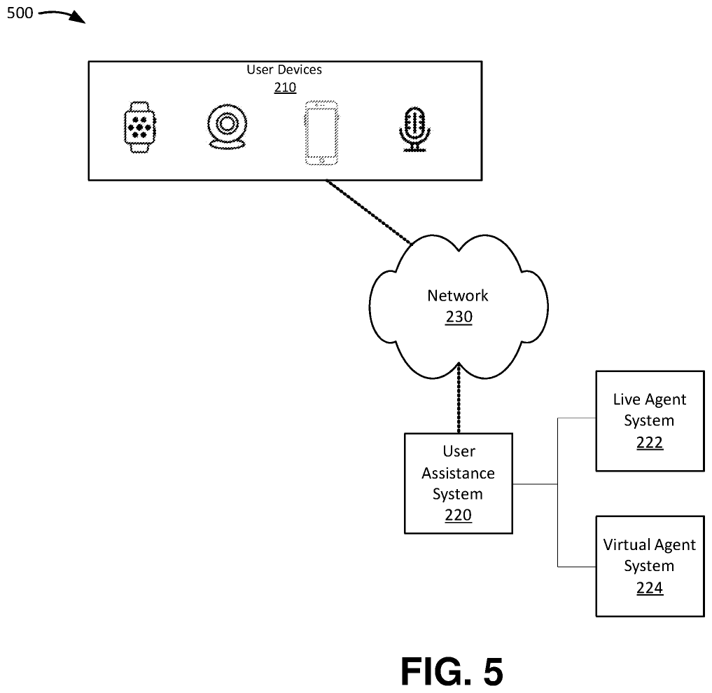

FIG. 5 shows an example environment in accordance with aspects of the present invention. As shown in FIG. 5, environment 500 may include one or more user devices 210, a user assistance system 220, and a network 230. In embodiments, one or more components in environment 500 may correspond to one or more components in the cloud computing environment of FIG. 2. In embodiments, one or more components in environment 500 may include the components of computer system/server 12 of FIG. 1.

The user device 210 may include a device capable of communicating via a network, such as the network 230. For example, the user device 210 may correspond to a mobile communication device (e.g., a smart phone or a personal digital assistant (PDA)), a portable computer device (e.g., a laptop or a tablet computer), a desktop computer, an accessory device (e.g., a smart wearable computing device) and/or another type of computing device. As used herein, user device 210 may correspond to a single device, or a combination of connected devices (e.g., either separate devices or integrated devices). In embodiments, a user device 210 may receive biometrics data via one or more integrated or separate and connected sensors/accessory devices. In some embodiments, the user device 210 may implement a support application for receiving support about a topic (e.g., via a chat platform, audio/video conferencing, remote screen sharing/control techniques, etc.).

The user assistance system 220 may include one or more computing devices (e.g., such as computer system/server 12 of FIG. 1) that implements a live agent system 222 and a virtual agent system 224. The live agent system 222 may include a platform for a live agent to provide support to a user. The virtual agent system 224 may include an application that provides automated support to the user. As described herein, the virtual agent system 224 may implement a business process when providing support to the user and may follow a particular path, of multiple possible paths, within the business process in which the particular path is based on emotional state data and/or user demographics.

The network 230 may include network nodes, such as network nodes 10 of FIG. 2. Additionally, or alternatively, the network 230 may include one or more wired and/or wireless networks. For example, the network 230 may include a cellular network (e.g., a second generation (2G) network, a third generation (3G) network, a fourth generation (4G) network, a fifth generation (5G) network, a long-term evolution (LTE) network, a global system for mobile (GSM) network, a code division multiple access (CDMA) network, an evolution-data optimized (EVDO) network, or the like), a public land mobile network (PLMN), and/or another network. Additionally, or alternatively, the network 230 may include a local area network (LAN), a wide area network (WAN), a metropolitan network (MAN), the Public Switched Telephone Network (PSTN), an ad hoc network, a managed Internet Protocol (IP) network, a virtual private network (VPN), an intranet, the Internet, a fiber optic-based network, and/or a combination of these or other types of networks.

The quantity of devices and/or networks in the environment 500 is not limited to what is shown in FIG. 5. In practice, the environment 500 may include additional devices and/or networks; fewer devices and/or networks; different devices and/or networks; or differently arranged devices and/or networks than illustrated in FIG. 5. Also, in some implementations, one or more of the devices of the environment 500 may perform one or more functions described as being performed by another one or more of the devices of the environment 500. Devices of the environment 500 may interconnect via wired connections, wireless connections, or a combination of wired and wireless connections.

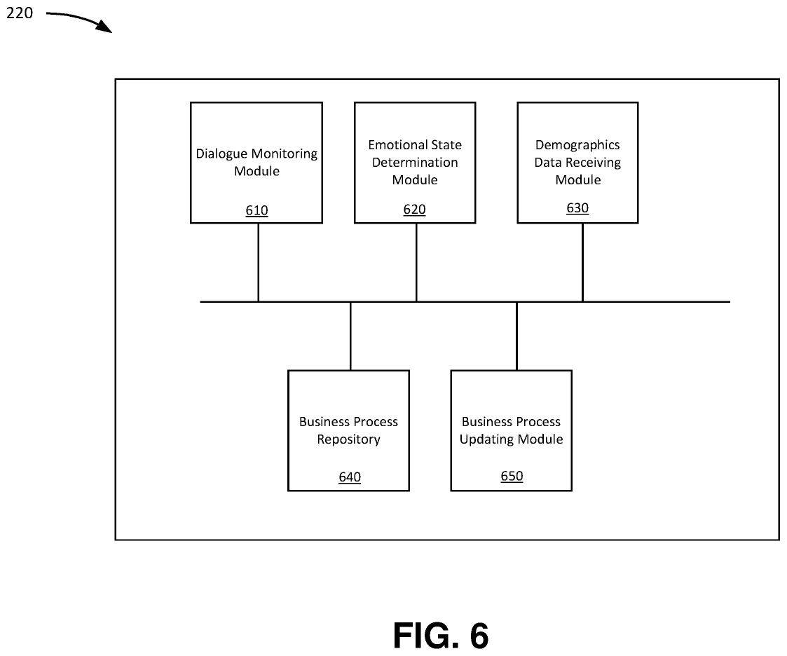

FIG. 6 shows a block diagram of example components of a user assistance system 220 in accordance with aspects of the present invention. As shown in FIG. 6, the user assistance system 220 may include a dialogue monitoring module 610, an emotional state determination module 620, a demographics data receiving module 630, a business process repository 640, and a business process updating module 650. In embodiments, the user assistance system 220 may include additional or fewer components than those shown in FIG. 6. In embodiments, separate components may be integrated into a single computing component or module. Additionally, or alternatively, a single component may be implemented as multiple computing components or modules.

The dialogue monitoring module 610 may include a program module (e.g., program module 42 of FIG. 1) that monitors a support dialogue (e.g., between a user and a live agent through the use of a user device 210 and a live agent system 222). In embodiments, the dialogue monitoring module 610 may monitor and record the steps taken within a business process by the live agent when providing the user with support assistance regarding a topic.

The emotional state determination module 620 may include a program module (e.g., program module 42 of FIG. 1) that determines the emotional state of the user based on biometrics data received from the user device 210 during the dialogue. In embodiments, the biometrics data may include pulse rate, perspiration data, eye behavior information (eye blinking patterns/timing data, moisture level data), visual biometrics data, behavioral biometrics data, audio biometrics data, or the like. In embodiments, the emotional state determination module 620 may derive the emotional state of the user using the emotional state data and one or more suitable cognitive emotional state determination techniques (e.g., biometrics analysis, voice analysis of the user's voice/tone, natural language processing of the user's dialogue, video/image analysis of the user/user facial expressions during the dialogue, etc.).

The demographics data receiving module 630 may include a program module (e.g., program module 42 of FIG. 1) that receives demographics data regarding the user. In embodiments, the demographics data receiving module 630 may receive the demographics information from the user via the user device 210 at any time during the dialogue. For example, the demographics data receiving module 630 may prompt the user for demographics information at the initiation of a support dialogue, or at another time during the dialogue.

The business process repository 640 may include a data storage device (e.g., storage system 34 of FIG. 1) that stores business processes used to provide a user with support about a topic. As described herein, the business process repository may store emotional state-based mapping information that maps a particular path, of multiple possible paths, to follow within the business process based on demographics data and/or user emotional state data (e.g., the emotional state of the user at each step of the business process during the dialogue, the aggregate/overall emotional state of the user during a support dialogue).

The business process updating module 650 may include a program module (e.g., program module 42 of FIG. 1) that updates the business process emotional state-based mapping information that maps (or generates the emotional state-based mapping information if the mapping information does not yet exist). In embodiments, the business process updating module 650 may update or generate the emotional state-based mapping information within the business process stored by the business process repository 640. The business process updating module 650 may update or generate the emotional state-based mapping information by mapping a particular path, of multiple possible paths, that a live agent followed within a business process during a support dialogue (e.g., based on the dialogue monitored by the dialogue monitoring module 610), and linking the mapped path with the emotional state of the user (e.g., as derived by the emotional state determination module 620), and/or the user's demographics (e.g., as received by the demographics data receiving module 630). In this way, the emotional state-driven mapping information includes information that links the particular path followed by a live agent to the emotional state and/or demographics of the user. As described herein, a virtual agent system 224 may use the emotional state-based mapping information (e.g., generated/updated by the business process updating module 650 and stored by the business process repository 640) to provide “life-like” support based on the user's emotional state for complex support topics in which the user's emotional state affects the path taken within the business process to provide the best suited support for the user.

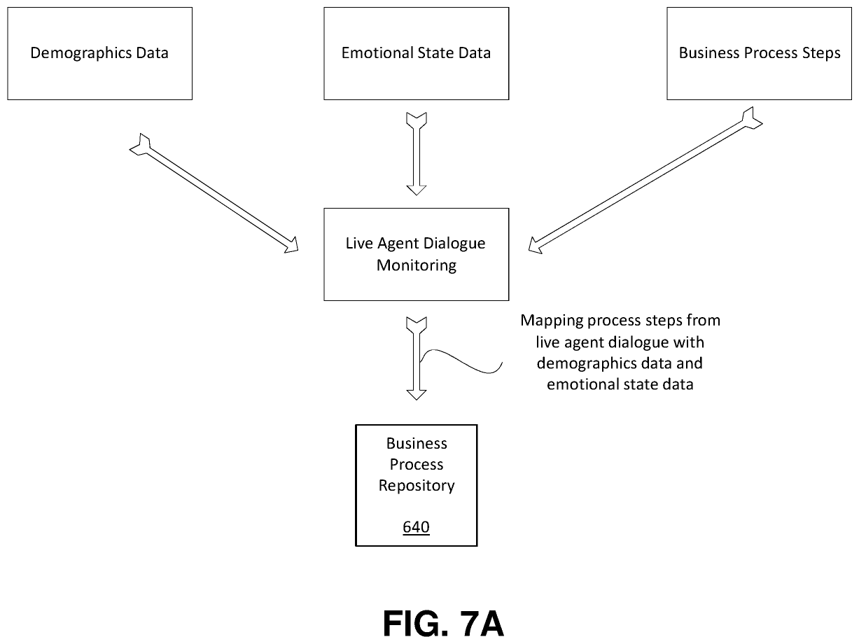

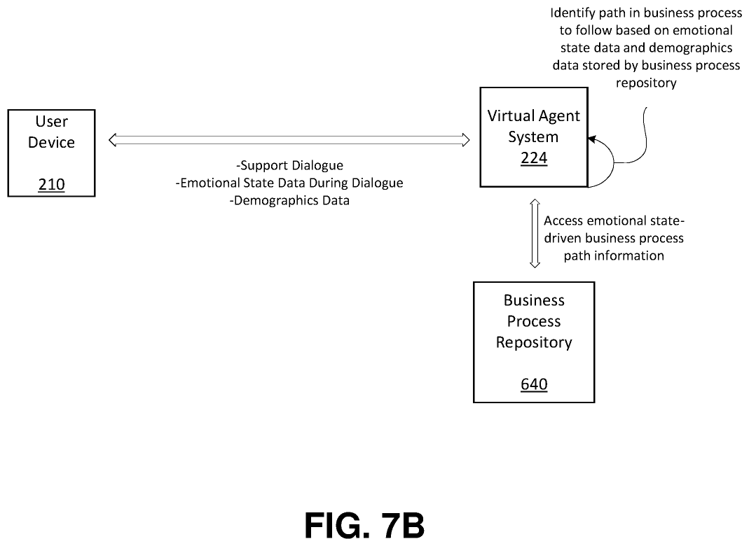

FIGS. 7A and 7B show example diagrams of a process for generating emotional state-based mapping information and adapting a virtual agent system using the emotional state-based mapping information. As shown in FIG. 7A, demographics data, emotional state data, and business process steps are received and/or monitored during live agent dialogue monitoring. The business process stored by the business process repository 640 is updated to include the mapped process steps from the live agent dialogue with the demographics data and the emotional state data.

Referring to FIG. 7B, the virtual agent system 224 may determine the emotional state and/or demographics data from a support dialogue (e.g., a new support dialogue or a support dialogue that was transferred from a live agent to the virtual agent system 224). Further, the virtual agent system 224 may access emotional state-driven business process path information from the business process repository 640, and identify the path in the business process to follow based on the emotional state data, the demographics data and the emotional state-driven business process path information stored by the business process repository 640. The virtual agent system 224 may follow the identified path in the business process in order to provide automated support with a similar support experience as a live agent by following the same path as did a live agent when providing live support to a prior user with similar demographics and emotional state.

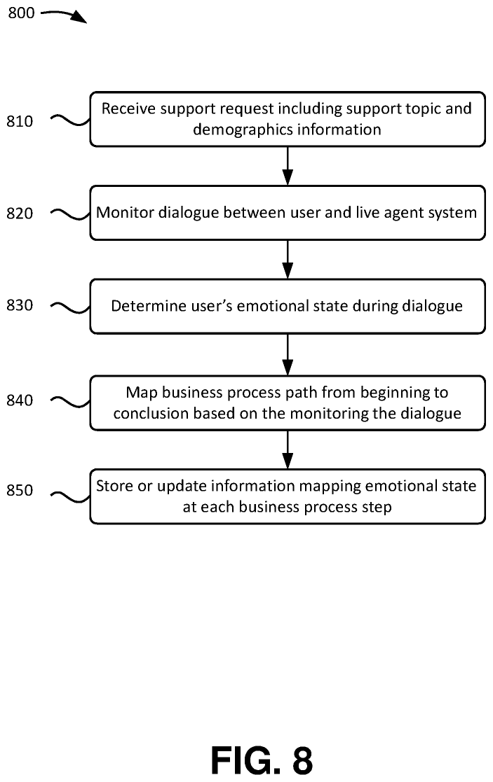

FIG. 8 shows an example flowchart of a process for generating emotional state-based mapping information. The steps of FIG. 8 may be implemented in the environment of FIG. 5, for example, and are described using reference numbers of elements depicted in FIG. 5. As noted above, the flowchart illustrates the architecture, functionality, and operation of possible implementations of systems, methods, and computer program products according to various embodiments of the present invention.

As shown in FIG. 8, process 800 may include receiving a support request including a support topic and demographics information (step 810). For example, as described above with respect to the dialogue monitoring module 610 and the demographics data receiving module 630, the user assistance system 220 may receive a support request from a user via a user device 210. The support request may include information identifying a topic (e.g., an initial utterance from which the user assistance system 220 may determine the request topic). Additionally, the support request may include the user's demographics information, or information that is linked to the user's demographic information (e.g., user profile/login information, etc.).

Process 800 may also include monitoring the dialogue between the user and live agent system (step 820). For example, as described above with respect to the dialogue monitoring module 610, the user assistance system 220 may monitor and store the dialogue between the user device and a live agent via the live agent system 222. The user assistance system 220 may monitor and record the steps taken within a business process by the live agent when providing the user with support assistance regarding a topic.

Process 800 may further include determining the user's emotional state during the dialogue (step 830). For example, as described above with respect to the emotional state determination module 620, the user assistance system 220 may determines the emotional state of the user based on biometrics data received from the user device 210 during the dialogue. In embodiments, the user assistance system 220 may derive the emotional state of the user using the emotional state data and one or more suitable cognitive emotional state determination techniques (e.g., biometrics analysis, voice analysis of the user's voice/tone, natural language processing of the user's dialogue, video/image analysis of the user/user facial expressions during the dialogue, etc.).

Process 800 may also include mapping the business process path from beginning to conclusion based on the monitoring the dialogue (step 840). For example, as described above with respect to the business process updating module 650, the user assistance system 220 may map or identify a path followed by the live agent within a business process as the dialogue progresses from the start to its conclusion.

Process 800 may further include storing or updating information mapping the emotional state at each business process step (step 850). For example, as described above with respect to the business process repository 640 and the business process updating module 650, the user assistance system 220 may update or generate the emotional state-based mapping information by monitoring the path that a live agent took within a business process during a support dialogue (e.g., based on the dialogue monitored by the dialogue monitoring module 610), the emotional state of the user (e.g., as derived by the emotional state determination module 620), and/or the user's demographics (e.g., as received by the demographics data receiving module 630).

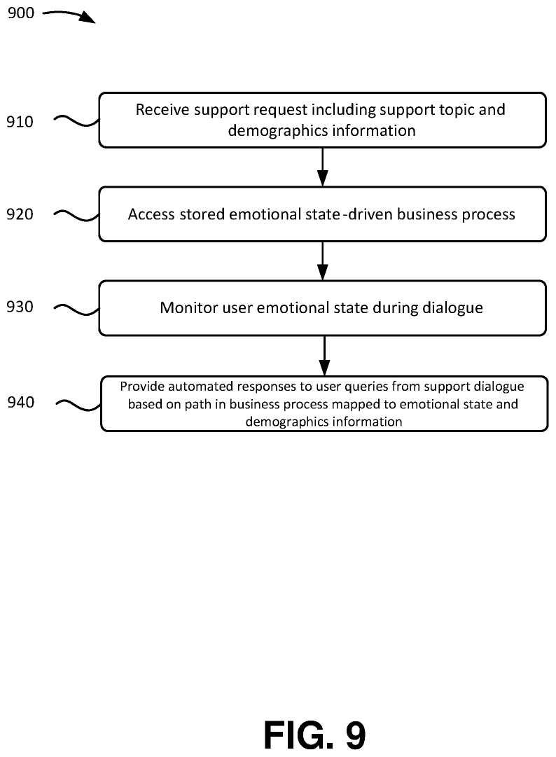

FIG. 9 shows an example flowchart of a process for adapting a virtual agent to provide support to a user based on the user's demographics and/or emotional state. The steps of FIG. 9 may be implemented in the environment of FIG. 5, for example, and are described using reference numbers of elements depicted in FIG. 5. As noted above, the flowchart illustrates the architecture, functionality, and operation of possible implementations of systems, methods, and computer program products according to various embodiments of the present invention.

As shown in FIG. 9, process 900 may include receiving a support request including a support topic and demographics information (step 910). For example, the virtual agent system 224 may receive a support request and demographics information (e.g., as part of a new support request from the user device 210 or when a support dialogue is transferred from the live agent system 222 to the virtual agent system 224).

Process 900 may also include accessing a stored emotional state-driven business process (step 920). For example, the virtual agent system 224 may access the emotional state-driven business process stored by the business process repository 640.

Process 900 may further include monitoring the user's emotional state during the dialogue (step 930). For example, as described above with respect to the emotional state determination module 620, the virtual agent system 224 may monitor the user's emotional state during the dialogue using one or more suitable cognitive emotional state determination techniques.

Process 900 may also include providing automated responses to user queries from the support dialogue based on a path in the business process that is mapped to the emotional state and the demographics information (step 940). For example, the virtual agent system 224 may provide automated responses in accordance with a particular path within the business process in which the path is determined based on the emotional state and the demographics information. In this way, the virtual agent system 224 may provide users with support in a similar manner as did a live agent, factoring in emotional state data and/or demographics data

In embodiments, a service provider could offer to perform the processes described herein. In this case, the service provider can create, maintain, deploy, support, etc., the computer infrastructure that performs the process steps of the invention for one or more customers. These customers may be, for example, any business that uses technology. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer system/server 12 (FIG. 1), can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system/server 12 (as shown in FIG. 1), from a computer-readable medium; (2) adding one or more computing devices to the computer infrastructure; and (3) incorporating and/or modifying one or more existing systems of the computer infrastructure to enable the computer infrastructure to perform the processes of the invention.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
monitoring, by a computing device, a support dialogue between a user device and a live agent system associated with the computing device;
determining, by the computing device, emotional state information associated with the support dialogue;
generating, by the computing device, emotional state-driven mapping information by mapping a particular path, of a plurality of possible paths, in a business process followed by the live agent system during a start of the support dialogue to a conclusion of the support dialogue and linking the particular path to the emotional state information;
storing, by the computing device, the emotional state-driven mapping information;
accessing, by the computing device, the emotional state-driven mapping information by a virtual agent system associated with the computing device;
providing, by the computing device, automated support by the virtual agent system for a future support dialogue using the emotional state-driven mapping information.

2. The method of claim 1, wherein the support dialogue is between a user using the user device and live agent using the live agent system.

3. The method of claim 2, wherein the emotional state information identifies an emotional state of the user during the support dialogue.

4. The method of claim 1, further comprising receiving demographics information, wherein the emotional state-driven mapping information links the demographics information to the particular path.

5. The method of claim 1, wherein the emotional state information is based on biometrics data received by the user device.

6. The method of claim 5, wherein the user device includes a wearable computing device, wherein the biometrics data includes at least one selected from the group consisting of:
pulse rate data;
perspiration data;
eye behavior;
visual biometrics data;
behavioral biometrics data; and
audio biometrics data.

7. The method of claim 1, wherein the determining the emotional state information from biometrics data includes at least one selected from the group consisting of:
biometrics analysis;
voice analysis;
natural language processing;
video/image analysis.

8. The method of claim 1, further comprising:
receiving a support request from the user device;
determining whether the emotional state-driven mapping information is available;
providing the automated support using the emotional state-driven mapping information when emotional state-driven mapping information is available; and
generate the emotional state-driven mapping information when the emotional state-driven mapping information is not available.

9. The method of claim 1, wherein the support dialogue takes place via a support platform including at least one selected from the group consisting of:
a text chat system;
a audio or video conference system; and
a remote screen sharing or control system.

10. The method of claim 1, wherein a service provider at least one of creates, maintains, deploys and supports the computing device.

11. The method of claim 1, wherein the monitoring the support dialogue, the determining the emotional state information, the generating the emotional state-driven mapping information, the storing the emotional state-driven mapping information, the accessing the emotional state-driven mapping information, and the providing the automated support are provided by a service provider on a subscription, advertising, and/or fee basis.

12. The method of claim 1, wherein the computing device includes software provided as a service in a cloud environment.

13. The method of claim 1, further comprising deploying a system for adapting the virtual agent system to provide the automated support based on the emotional stated-driven information, comprising providing a computer infrastructure operable to perform the monitoring the support dialogue, the determining the emotional state information, the generating the emotional state-driven mapping information, the storing the emotional state-driven mapping information, the accessing the emotional state-driven mapping information, and the providing the automated support.

14. A computer program product for adapting a virtual agent system to provide automated support based on emotional stated-driven information, the computer program product comprising a computer readable storage medium having program instructions embodied therewith, the program instructions executable by a computing device to cause the computing device to:
monitor a support dialogue between a user device and a live agent system associated with the computing device;
determine emotional state information associated with the support dialogue;
generate emotional state-driven mapping information by mapping a particular path, of a plurality of possible paths, in a business process followed by the live agent system during a start of the support dialogue to a conclusion of the support dialogue and linking the particular path to the emotional state information;
store the emotional state-driven mapping information;
transfer the support dialogue to a virtual agent system associated with the computing device;
access the emotional state-driven mapping information by a virtual agent system; and
provide automated support by the virtual agent system for the transferred support dialogue using the emotional state-driven mapping information.

15. The computer program product of claim 14, wherein the support dialogue is between a user using the user device and live agent using the live agent system.

16. The computer program product of claim 14, wherein the program instructions further cause the user device to receive demographics information, wherein the emotional state-driven mapping information links the demographics information to the particular path.

17. The computer program product of claim 14, wherein the emotional state information is based on biometrics data received by the user device, wherein the user device includes a wearable computing device, wherein the biometrics data includes at least one selected from the group consisting of:
pulse rate data;
perspiration data;
eye behavior;
visual biometrics data;
behavioral biometrics data; and
audio biometrics data.

18. The computer program product of claim 14, wherein the determining the emotional state information from biometrics data includes at least one selected from the group consisting of:
biometrics analysis;
voice analysis;
natural language processing;
video/image analysis.

19. A system comprising:
a CPU, a computer readable memory and a computer readable storage medium associated with a computing device;
program instructions to receive a support request from a user device, wherein the support request identifies a support topic;
program instructions to determine whether emotional state-driven mapping information for a business process is available for the support topic;
program instructions to generate the emotional state-driven mapping information when the emotional state-driven mapping information is not available, wherein the generating comprises:
program instructions to route the support request to a live agent system,
program instructions to determine emotional state information associated with the support dialogue, and
program instructions to map a particular path, of a plurality of possible paths, in the business process followed by the live agent system during a start of the support dialogue to a conclusion of the support dialogue and linking the particular path to the emotional state information; and
program instructions to provide, when the emotional state-driven mapping information is available, automated support using the emotional state-driven mapping information and based on emotional state data received from the user device,
wherein the program instructions are stored on the computer readable storage medium for execution by the CPU via the computer readable memory.

20. The system of claim 19, further comprising program instructions to receive demographics information, wherein the emotional state-driven mapping information links the demographics information to the particular path.
